SEQUENCE LISTING (12 sequences)

SEQ ID NO: 1 - DNA, 23 bp, synthetic construct
  source 1..23: mol_type = other DNA
gtacacctacccgtcaccggagg

SEQ ID NO: 2 - DNA, 18 bp, synthetic construct
  source 1..18: mol_type = other DNA
caattaaccctcactaaa

SEQ ID NO: 3 - DNA, 47 bp, synthetic construct
  source 1..47: mol_type = other DNA
aggagaccgctataaccggtttgaatttatgaacaacaataccagcc

SEQ ID NO: 4 - DNA, 59 bp, synthetic construct
  source 1..59: mol_type = other DNA
gttatttacagaagttggaaggctggtattgttgttcaaatgtagacgtactcgacatg

SEQ ID NO: 5 - DNA, 52 bp, synthetic construct
  source 1..52: mol_type = other DNA
ggttttacccattcatgtcgagtacgtctacatttgaacaacaataccagcc

SEQ ID NO: 6 - DNA, 58 bp, synthetic construct
  source 1..58: mol_type = other DNA
atgaacttgcttgctgtcaaacttctgagttggtttattcatcattatcatcatcatc

SEQ ID NO: 7 - DNA, 55 bp, synthetic construct
  source 1..55: mol_type = other DNA
ttcctcgggcagagaaactcgcaggcaacttggaaggttcacgaaatctttaatg

SEQ ID NO: 8 - DNA, 50 bp, synthetic construct
  source 1..50: mol_type = other DNA
ccctctgtgagggccgattatgcaggcctagaaaaagaccaaacggtgac

SEQ ID NO: 9 - DNA, 50 bp, synthetic construct
  source 1..50: mol_type = other DNA
tccactcccccatttttatccggatctctgcacgcaaaagaaaaccttcg

SEQ ID NO: 10 - DNA, 50 bp, synthetic construct
  source 1..50: mol_type = other DNA
tgggctcaaggtgacaaggtcctcgaaaatagggccgcaaattaaagcct

SEQ ID NO: 11 - DNA, 50 bp, synthetic construct
  source 1..50: mol_type = other DNA
tgggacgctcgaaggctttaatttgcggccctattttcgaggaccttgtc

SEQ ID NO: 12 - DNA, 57 bp, synthetic construct
  source 1..57: mol_type = other DNA
ctggcaaacggcgctctccaaggcacaaacttactatttttcagtcttaaatgcttc

(19) United States
(12) Patent Application Publication
(10) Pub. No.: US 2026/0159804 A1
(43) Pub. Date: Jun. 11, 2026

(54) CO2-FIXING MICROORGANISM

(71) Applicant: KYUNGPOOK NATIONAL UNIVERSITY INDUSTRY-ACADEMIC COOPERATION FOUNDATION, Daegu (KR)

(72) Inventors: Soo Rin Kim, Daegu (KR); Sujeong Park, Daegu (KR)

(73) Assignee: KYUNGPOOK NATIONAL UNIVERSITY INDUSTRY-ACADEMIC COOPERATION FOUNDATION, Daegu (KR)

(21) Appl. No.: 19/394,823

(22) Filed: Nov. 19, 2025

(30) Foreign Application Priority Data
Dec. 9, 2024 (KR) 10-2024-0181707

(51) Int. Cl. C12N 1/18; C07K 14/195; C07K 14/395; C12N 9/12; C12N 9/88; C12P 7/06; C12R 1/865

(52) U.S. Cl. CPC C12N 1/185; C07K 14/195; C07K 14/395; C12N 9/1205; C12N 9/88; C12P 7/06; C12Y 207/01019; C12Y 401/01039; C12R 2001/865

(57) ABSTRACT

The present disclosure relates to a microorganism that fixes carbon dioxide, presenting a new strategy capable of contributing to achieving carbon neutrality in bioprocesses through development of a CO₂ fixing yeast using S. cerevisiae, wherein, to address the redox imbalance problem and increase CO₂ fixation efficiency, a form-I RuBisCO-based CO₂ fixation pathway was established in a xylose isomerase-based xylose metabolic strain, the fermentation rate and productivity were remarkably improved by deleting the endogenous reductase genes GRE3 and YPR1, which induce xylitol accumulation, resulting in a significant increase in a yield of ethanol production along with efficient CO₂ fixation, the finally developed SJ11 YΔGΔ strain had significantly reduced xylitol accumulation, greatly improved fermentation rate, and maximized CO₂ fixation efficiency and ethanol productivity compared to existing strains.

### CROSS-REFERENCE TO RELATED APPLICATION

This application claims the benefit of Korean Patent Application No. 10-2024-0181707 filed on Dec. 9, 2024, in the Korean Intellectual Property Office, the entire disclosure of which is incorporated herein by reference for all purposes.

### SEQUENCE LISTING

This application contains a Sequence Listing submitted via USPTO Patent Center and hereby incorporated by reference in its entirety. The Sequence Listing is named 2280-622.xml, created on Nov. 19, 2025, and 11,463 bytes in size.

### BACKGROUND

### 1. Field of the Invention

The present disclosure relates to a carbon dioxide (CO₂)-fixing microorganism and, more specifically, to a recombinant microorganism that produces ethanol from carbon dioxide by maximally expressing a carbon dioxide fixing enzyme.

### 2. Description of the Related Art

Unpredictable climate change caused by global warming threatens humanity and the ecosystem. Carbon dioxide (CO₂) accounts for the largest proportion of greenhouse gases, with 34 billion tons emitted every year due to the increased use of fossil fuels. To alleviate the problem of climate change caused by global warming, a goal of achieving carbon neutrality by 2050 has been adopted, and reduction in CO₂ emissions from industrial processes is essential for the goal. Accordingly, converting fossil fuel-based processes to bioprocesses is being proposed as a strategy for reducing carbon dioxide emissions. Saccharomyces cerevisiae, which is yeast, is widely used in the production of biofuels and bio-based products since it ensures easiness in gene edition using Cas9 and capable of efficiently producing a variety of products. For sustainable yeast-based bioprocesses, S. cerevisiae strains have also been improved to metabolize inedible wastes such as lignocellulosic biomass. However, CO₂ emission is inevitable even in microbial fermentation-based bioprocesses, and in particular, S. cerevisiae strains are reported to have higher CO₂ emission than other yeast species.

Therefore, research is being conducted to develop S. cerevisiae strains capable of fixing CO₂ to achieve carbon neutrality in bioprocesses. Several studies are focusing on the development of CO₂ fixing yeast through introduction of ribulose-1,5-bisphosphate carboxylase/oxygenase (RuBisCO), a carbon dioxide fixing enzyme in the Calvin-Benson-Bassham pathway, into yeast. In particular, in the case of the xylose-metabolizing CO₂ fixing yeast SJ03 developed in a previous study, ethanol productivity has been improved through carbon dioxide fixation, and a decrease in CO₂ emissions was observed during actual fermentation. However, in the SJ03 strain, there was an issue regarding redox imbalance due to the use of cofactors in the xylose metabolism process based on the xylose oxidoreductase pathway, leading to excessive accumulation of xylitol, a byproduct, to result in a loss of carbon source (FIG. 1(a)). This redox imbalance causes negative outcomes such as degradation in microbial fermentation rate or reduction in productivity, such that it is essential to address this problem and develop strains with improved CO₂ fixation efficiency.

### SUMMARY

### Problem to be Solved by the Invention

The present disclosure provides a recombinant Saccharomyces cerevisiae SJ11 strain which is deposited under KCTC 15918BP and has increased CO₂ fixation efficiency based on a xylose isomerase pathway, an SJ11 GRE3Δ strain, SJ11 YPR1Δ strain, or SJ11 YΔGΔ strain with one or more of GRE3 and YPR1 genes deleted from a genome of the SJ11 strain, and a method of improving ethanol production including culturing the strain.

### Means for Solving the Problem

To address the above problem, the present disclosure provides a recombinant Saccharomyces cerevisiae SJ11 strain which is deposited under KCTC 15918BP and has increased CO₂ fixation efficiency based on a xylose isomerase pathway.

In addition, the present disclosure provides a recombinant Saccharomyces cerevisiae SJ11 GRE3Δ strain with GRE3 deleted from a genome of the SJ11 strain.

In addition, the present disclosure provides a recombinant Saccharomyces cerevisiae SJ11 YPR1Δ strain with YPR1 deleted from a genome of the SJ11 strain.

In addition, the present disclosure provides a recombinant Saccharomyces cerevisiae SJ11 YΔGΔ strain with GRE3 and YPR1 deleted from a genome of the SJ11 strain.

In addition, the present disclosure provides a method of improving ethanol production, including culturing the strain.

### Effects of the Invention

The present disclosure relates to a microorganism that fixes carbon dioxide, presenting a new strategy that may contribute to achieving carbon neutrality in bioprocesses through development of a CO₂ fixing yeast using S. cerevisiae. To address the redox imbalance problem and increase CO₂ fixation efficiency, a form-I RuBisCO-based CO₂ fixation pathway was established in a xylose isomerase-based xylose metabolic strain. The strain developed showed improved ethanol productivity compared to a control group through CO₂ fixation. Additionally, the fermentation rate and productivity were remarkably improved by deleting endogenous reductase genes GRE3 and YPR1, which induce xylitol accumulation, resulting in a significant increase in a yield of ethanol production along with efficient CO₂ fixation. The finally developed SJ11 YΔGΔ strain exhibited significantly reduced xylitol accumulation, greatly improved fermentation rate, and maximized CO₂ fixation efficiency and ethanol productivity compared to existing strains. These results suggest that it may be a strategy for achieving carbon neutrality by addressing redox imbalances and minimizing byproduct accumulation in microbial fermentation-based bioprocesses.

### BRIEF DESCRIPTION OF THE DRAWINGS

FIG. 1 shows construction of a CO₂-fixation pathway in a xylose-metabolizing strain. (a) CO₂-fixation-based xylose oxidoreductase pathway, (b) CO₂-fixation-based xylose isomerase pathway. XR, xylose reductase; XDH, xylitol dehydrogenase; XKS; xylulokinase; PRK, phosphoribulokinase; RuBisCO, ribulose-1,5-bisphosphate carboxylase-oxygenase.

FIG. 2 shows schematic diagrams for constructing a CO₂-fixation pathway in a δXI strain. (a) A schematic diagram for constructing a gene expression cassette, (b) A schematic diagram for constructing a CO₂-fixation pathway.

FIG. 3 shows results of comparing various characteristics of a control strain (XI) and a constructed strain (SJ11). (a) Xylose consumption, (b) Xylitol production, (c) Ethanol production. All experiments were performed in triplicate biological replicates, and error bars represent standard deviation.

FIG. 4 shows effects of deletion of endogenous reductase and dehydrogenase. (a) Deletion target gene, (b) Xylose consumption, (c) Xylitol production, (d) Ethanol production. All experiments were performed in triplicate biological replicates, and error bars represent standard deviation.

FIG. 5 shows effects of deletion of YPR1 and GRE3. (a) Growth profile, (b) Xylose consumption, (c) Xylitol production, (d) Ethanol production. All experiments were performed in triplicate biological replicates, and error bars represent standard deviation.

### DETAILED DESCRIPTION

In the present disclosure, a S. cerevisiae strain with improved CO₂ fixation and fermentation efficiency was developed by resolving an issue regarding the redox imbalance. A CO₂-fixing yeast strain was developed by introducing a RuBisCO-based CO₂-fixation pathway into a xylose isomerase-based xylose metabolic strain that does not utilize a cofactor, and genes related to endogenous xylitol production were additionally deleted to reduce byproduct production and improve CO₂-fixation efficiency. The present disclosure was completed by discovering improvement in CO₂ fixation and fermentation efficiency through comparison of phenotypic changes.

The present disclosure provides a recombinant Saccharomyces cerevisiae SJ11 strain, which is deposited under accession number KCTC 15918BP and has improved CO₂ fixation efficiency based on a xylose isomerase pathway.

Preferably, the strain may include cbbL1, cbbS1, and cfxP1 derived from Ralstonia eutropha H16, and HSP60 and HSP10 derived from Saccharomyces cerevisiae D452-2 with their signal peptides removed, introduced into its genome.

In addition, the present disclosure provides a recombinant Saccharomyces cerevisiae SJ11 GRE3Δ strain with GRE3 deleted from a genome of the SJ11 strain.

In addition, the present disclosure provides a recombinant Saccharomyces cerevisiae SJ11 YPR1Δ strain with YPR1 deleted from a genome of the SJ11 strain.

In addition, the present disclosure provides a recombinant Saccharomyces cerevisiae SJ11 YΔGΔ strain with GRE3 and YPR1 deleted from a genome of the SJ11 strain.

In addition, the present disclosure provides a method of improving ethanol production, including culturing the strain.

In the present disclosure, the CRISPR-Cas9 system may be used to introduce a gene into the genome of the Saccharomyces cerevisiae strain. In the present disclosure, a gene may be introduced into the genome of a Saccharomyces cerevisiae strain based on the CRISPR/Cas9 system and introduced into an intergenic region of the Saccharomyces cerevisiae strain.

In the present disclosure, the cbbL1 and cbbS1, which are RuBisCO genes derived from Ralstonia eutropha H16, may have NCBI accession no. OP654634.1 and OP654635.1, respectively.

In the present disclosure, the cfxP1 is a PRK gene that is essential for RuBisCO substrate production derived from Ralstonia eutropha H16, with NCBI accession no. CP039288.1.

In the present disclosure, the tHSP60 and tHSP10 are HSP60 and HSP10 with a mitochondrial signal peptide derived from Saccharomyces cerevisiae D452-2 deleted, and NCBI accession no. of HSP60 and HSP10 may be NC_001144.5 and NC_001147.6, respectively.

In the present disclosure, the GRE3 and YPR1 are endogenous reductase genes of Saccharomyces cerevisiae, and NCBI accession no. may be NC_001140.6 and NC_001136.10, respectively.

Hereinafter, the present disclosure will be described in detail according to examples that do not limit the present disclosure. It should be noted that the following examples of the present disclosure are intended only to illustrate the present disclosure and do not limit or restrict the scope of the present disclosure. Therefore, it is interpreted that what may be easily inferred by those skilled in the art to which the present disclosure pertains from the detailed description and examples of the present disclosure falls within the scope of the present disclosure.

### EXPERIMENTAL EXAMPLE

The following Experimental Examples are intended to provide Experimental Examples commonly applied to each Example according to the present disclosure.

### 1. Strains and Media

The strains used herein are summarized in Table 1. S. cerevisiae strains were inoculated into YP (10 g/L yeast extract, 20 g/L peptone) liquid medium containing 20 g/L glucose and pre-cultured for 24 hours at 30° C. and 250 rpm. Escherichia coli Top10 strain (Invitrogen, Carlsbad, CA, USA) was used for amplification of guide RNA plasmids. E. coli strains were cultured at 37° C. and 250 rpm in Luria-Bertani medium (5 g/L yeast extract, 10 g/L tryptone, 10 g/L NaCl) containing 100 nag/mL ampicillin.

TABLE 1

| Strains | Description/relevant genotype ¹⁾ |
|---|---|
| D452-2 | Wild-type; Matα leu2 his3 ura3 |
| δXI | Xylose-utilizing engineered strain; |
| | D452-2 ald6Δ::P_{TDH3}-XYLA-T_{TDH3}int#1::P_{TEF1}-XYL3-T_{TEF1} |
| | leu2::LEU2 pYS-δXI with pho13Δ |
| SJ11 | Xylose and CO₂ utilizing-engineered strain; |
| | δXIint#11::P_{CCW12}-opcbbL1-T_{CYC1}-P_{TDH3}-opcbbS1-T_{ADH4} |
| | int# 12::P_{TEF1}-cfxP1-T_{TEF1}int#6::P_{PGK1}-tHSP60-T_{ADH7}-P_{FBA1}- |
| | tHSP10-T_{MDH1} |
| δXI YΔGΔ | δXI YPRIΔ GRE3Δ |
| SJ11 YΔGΔ | SJ11 YPRIΔ GRE3Δ |
| pRS41N-Cas9 | pRS41N plasmid containing a natNT marker and Cas9 |
| pRS42H-cg#1 | pRS42H plasmid containing a hph marker and gRNA for created 23-bp sequence |
| | (5′-GTACACCTACCCGTCACCGGAGG; SEQ ID NO: 1) |
| pRS42H-INT#6 | pRS42H plasmid containing a hph marker and gRNA for |
| | integration#6 (INT#6) |
| pRS42H-INT#11 | pRS42H plasmid containing a hph marker and gRNA for |
| | integration#11 (INT#11) |
| pRS42H-INT#12 | pRS42H plasmid containing a hph marker and gRNA for |
| | integration#12 (INT#12) |

¹⁾ cbbL1, cbbS1, and cfxP1 are derived from Ralstonia eutropha H16; Mitochondrial signal peptide-deleted HSP60 and HSP10 (tHSP60 and tHSP10) are derived from Saccharomyces cerevisiae D452-2.

### 2. Construction of Transformed Strains Using CRISPR-Cas9

Yeast strain improvement using Cas9 genetic scissors was performed based on previously published methods. For expression of Cas9 protein, the pRS41N-Cas9 plasmid was introduced into S. cerevisiae and selected on YPD solid medium containing cloNAT.

The plasmids and primers used herein are summarized in Tables 1 and 2. The guide RNA recognition sequence was designed using CHOPCHOP software (http://chopchop.cbu.uib.no/), and the guide RNA plasmid was constructed using the fast cloning method. Amplification was conducted by PCR using the pRS42H plasmid as a template, and the gRNA recognition sequence was determined using the T3 universal primer (5′-CAATTAACCCTCACTAAA-3′, SEQ ID NO: 2) before use in yeast transformation.

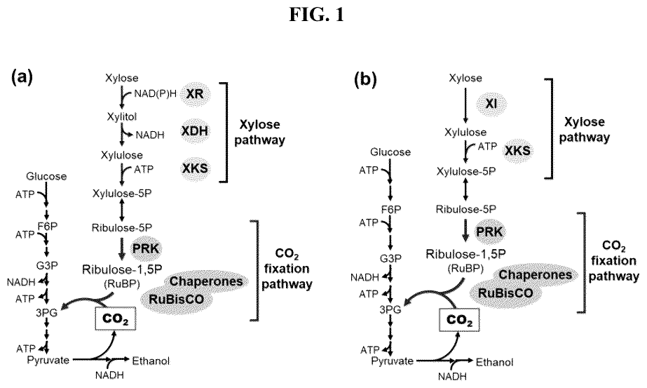

The cbbL1, cbbS1, and cfxP1 genes from Ralstonia eutropha H16 and the mitochondrial signal peptide deleted HSP60 and HSP10 genes, which are endogenous genes of S. cerevisiae, were amplified by PCR respectively and then introduced into the cg #1 sequence of the expression cassette (int #1::_{P}CCW12-cg #1-_{T}CYC1, int #6::_{P}TDH3-cg #1-_{T}TDH3, int #1::_{P}TEF1-cg #1-TTEF1, int #1::_{P}PGK1-cg #1-_{T}PGK1, and int #1::_{P}FBA1-cg #1-_{T}CYC1) which is introduced into D452-2 int #1 using Cas9 gene scissors (FIG. 1(A)). Each of the constructed expression cassettes was amplified by PCR using the primers listed in Table 2 to be introduced into the δXI strain that metabolizes xylose (FIG. 1(B)). The finally constructed strains were selected on YPD solid medium containing 100 μg/mL cloNAT and 300 μg/mL hygromicin B, and the insertion of each expression cassette was detected via colony PCR.

TABLE 2

| Primers | Sequences (5′-) | Description |
|---|---|---|
| Kim881 | AGGAGACCGCTATAACCGGTTTGAATTTA | Donor_INT#6_P_{PGK1}_F |
| | TGAACAACAATACCAGCC (SEQ ID NO: 3) | |
| Kim1407 | GTTATTTACAGAAGTTGGAAGGCTGGTATTGT | Donor_T_{ADH7}_R |
| | TGTTCA AATGTAGACGTACTCGACATG (SEQ | |
| | ID NO: 4) | |
| Kim1408 | GGTTTTACCCATTCATGTCGAGTACGTCTACAT | Donor_P_{FBA1}_R |
| | T TGAACAACAATACCAGCC (SEQ ID NO: 5) | |
| Kim1363 | ATGAACTTGCTTGCTGTCAAACTTCTGAGTTG | Donor_INT#6_T_{MDH1}_R |
| | GTTTATTCATCATTATCATCATCATC (SEQ ID | |
| | NO: 6) | |
| Kim1415 | TTCCTCGGGCAGAGAAACTCGCAGGCAACTTG | Donor_INT#4_P_{TEF1}_F |
| | GAAGGTTCACGAAATCTTTAATG (SEQ ID NO: | |
| | 7) | |
| Kim1416 | CCCTCTGTGAGGGCCGATTATGCAGGCCTAGA | Donor_INT#4_T_{TEF1}_R |
| | AAAAGACCAAACGGTGAC (SEQ ID NO: 8) | |
| Kim760 | TCCACTCCCCCATTTTTATCCGGATCTCTG | Donor_INT#11_P_{CCW12}_F |
| | CACGCAAAAGAAAACCTTCG (SEQ ID NO: 9) | |
| Kim761 | TGGGCTCAAGGTGACAAGGTCCTCGAAAATAG | Donor_T_{CYC1}_R |
| | GGCCGCAAATTAAAGCCT (SEQ ID NO: 10) | |
| Kim762 | TGGGACGCTCGAAGGCTTTAATTTGCGGCC | Donor_P_{TDH3}_F |
| | CTATTTTCGAGGACCTTGTC (SEQ ID NO: 11) | |
| Kim1406 | CTGGCAAACGGCGCTCTCCAAGGCACAAACTT | Donor_INT#11_T_{ADH4}_R |
| | ACTATTTTTCAGTCTTAAATGCTTC (SEQ ID NO: | |
| | 12) | |

### 3. Anaerobic Fermentation

S. cerevisiae strains were pre-cultured in YP (10 g/L yeast extract, 20 g/L peptone) liquid medium containing 20 g/L glucose. After pre-culture, only cells were recovered at 0.5 g DCW/L (g dry cell weight/L) and inoculated into YP medium containing 40 g/L xylose for fermentation. After inoculation inside an anaerobic chamber from which oxygen was removed for fermentation under anaerobic conditions, the inlet of a 125-mL serum bottle was sealed with a rubber stopper. Fermentation was carried out under conditions of 30° C. and 130 rpm.

### 4. HPLC Analysis

Measurement of xylose, xylitol, glycerol, and ethanol concentrations was analyzed using high-performance liquid chromatography (HPLC, Agilent Technologies, 1260 series, USA). A Rezex-ROA Organic Acid H+ (8%) (150 mm×4.6 mm) column (Phenomenex Inc., Torrance, CA, USA) and RI detector were used, and 0.005 N H₂SO₄ was used as the mobile phase. The sample was analyzed by eluting the mobile phase at a rate of 0.6 mL/min at 50° C.

### <Example 1> Construction of a CO₂ Fixing Yeast Based on Xylose Isomerase

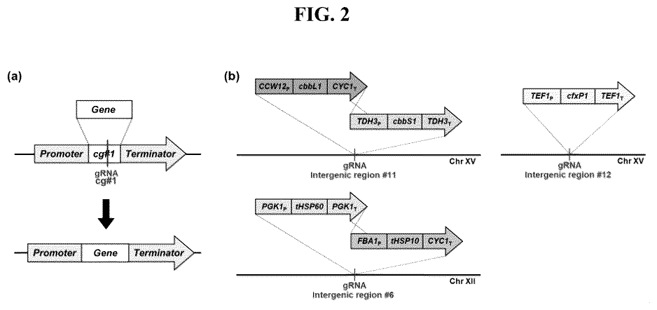

In the present disclosure, a form-I RuBisCO-based CO₂ fixation pathway, whose normal operation has been observed in S. cerevisiae, was introduced into the xylose isomerase-based xylose metabolism strain δXI developed in a previous study. Expression cassettes of the RuBisCO genes (cbbL1, cbbS1) derived from Ralstonia eutropha H16 and the PRK gene (cfxP1) essential for RuBisCO substrate production were introduced into the δXI strain to establish a CO₂ fixation pathway. For form-I RuBisCO, HSP60 and HSP10, endogenous chaperones of S. cerevisiae, have been reported to assist in the folding of RuBisCO proteins. To improve the folding of cytoplasmic RuBisCO, expression cassettes of HSP60 and HSP10 (tHSP60 and tHSP10) with the signal peptide removed were also introduced together into the δXI strain to ultimately construct the SJ11 strain (FIG. 2).

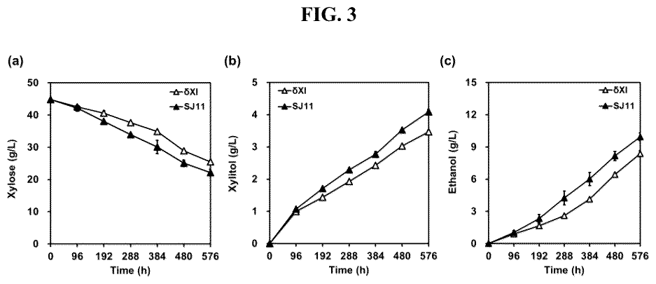

Since the operation of the CO₂ fixation pathway in yeast has been reported to increase the yield of ethanol production, the phenotypic changes of the S. cerevisiae CO₂ strain through anaerobic fermentation were compared with the control δXI strain (FIG. 3). Both the δXI strain and the SJ11 strain were unable to metabolize all xylose for up to 576 hours and showed slow xylose fermentation rates. Both the δXI strain and the SJ11 strain showed reduced production of xylitol, a by-product, compared to the strains based on the xylose oxidoreductase pathway developed in a previous study, indicating that the issue of redox imbalance due to cofactor imbalance was partially resolved through xylose isomerase-based xylose metabolism. Additionally, in the case of the CO₂ strain, the ethanol concentration and yield increased by 18.9% and 9.7%, respectively, compared to the control, and these phenotypic changes imply the normal operation of the CO₂ fixation pathway.

### <Example 2> Reduction of by-Product Accumulation Through Deletion of Endogenous Reductase and Dehydrogenase

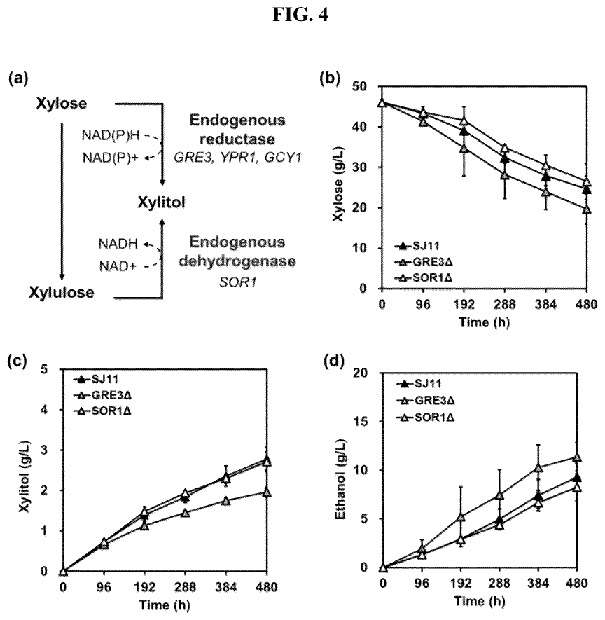

A decrease in xylitol production was detected through xylose metabolism based on a xylose isomerase pathway. However, it was found that xylitol still accumulated, which is presumed to be due to a nonspecific enzymatic reaction by endogenous reductase or dehydrogenase in yeast. To verify this, the effects on the SJ11 strain developed by deleting the GRE3 gene, which is an aldose reductase coding gene known to be involved in xylitol production, and the SOR1 gene, which codes for sorbitol dehydrogenase, were observed through anaerobic fermentation (FIG. 4(a)). When GRE3 was deleted, xylitol production was found to be significantly reduced compared to the SJ11 strain, and xylose consumption and ethanol production also increased (FIG. 4(b)-(d)). On the other hand, no significant phenotypic difference was observed when SOR1 was deleted, and these results show that the fermentation efficiency of the SJ11 strain was improved through the removal of endogenous reductase.

### <Example 3> Additional Selection and Deletion of Endogenous Reductase-Related Genes to Improve the Fermentation Efficiency of CO₂ Fixing Strains

It was determined that deletion of the endogenous reductase gene involved in xylitol accumulation had a positive effect on improvement of the carbon dioxide fixation and fermentation efficiency of the SJ11 strain through deletion of the GRE3 gene. Therefore, referring to previous studies, the endogenous reductase-related genes GRE3, YPR1, and GCY1, which may affect the reduction of xylitol accumulation, were additionally selected and deleted to identify the phenotypic changes depending on the deletion of each gene. Ethanol concentration and production yield were high in the order of SJ11 GRE3Δ, SJ11 YPR1Δ, and SJ11 GCY1Δ, and it was found that there was no significant difference in xylitol and ethanol concentration and yield of the SJ11 GCY1Δ strain compared to the SJ11 strain (Table 3). These results suggest that deletion of YPR1 and GRE3 may be a strategy for improving carbon dioxide fixation and fermentation efficiency in the SJ11 strain.

TABLE 3

| | | Product titers (g/L) | | | |
|---|---|---|---|---|---|
| Strains | Growth rate (g/L-h) | Xylitol | Ethanol | Xylitol yield (g/g) | Ethanol yield (g/g) |
| SJ11 | 1.77 ± 0.33 | 3.79 ± 0.10 | 10.69 ± 0.87 | 0.15 ± 0.01 | 0.43 ± 0.02 |
| SJ11 GRE3Δ | 1.98 ± 0.08 | 2.33 ± 0.33 | 13.37 ± 0.66 | 0.08 ± 0.01 | 0.47 ± 0.01 |
| SJ11 YPR1Δ | 1.59 ± 0.22 | 2.78 ± 0.13 | 10.73 ± 0.50 | 0.12 ± 0.00 | 0.45 ± 0.02 |
| SJ11 GCY1Δ | 1.43 ± 0.37 | 3.40 ± 0.18 | 9.90 ± 0.93 | 0.15 ± 0.02 | 0.43 ± 0.01 |

### <Example 4> Maximization of the Fermentation Efficiency of CO₂ Fixing Strains Through Additional Deletion of Endogenous Reductase-Related Genes

Previous results have shown that deletion of endogenous reductase genes YPR1 and GRE3, which are involved in xylitol accumulation, affected the improvement of ethanol productivity and the reduction of xylitol accumulation in SJ11. To maximize the CO₂ fixation and fermentation efficiency of the SJ11 strain, the SJ11 YΔGΔ strain was finally created, in which the YPR1 and GRE3 genes were deleted together.

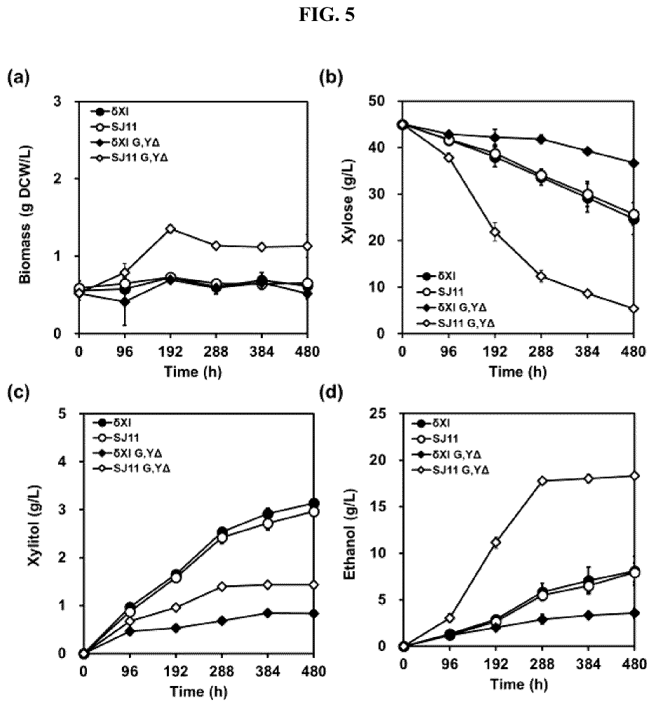

Unlike previous strains, the SJ11 YΔGΔ strain showed a much faster xylose consumption rate, and its growth rate was improved by 82.3% compared to the δXI strain and 73.8% compared to the CO₂ strain (FIG. 5(a) and Table 4). The δXI YΔGΔ strain, which is the control strain of the SJ11 YΔGΔ strain, showed a significant decrease in xylitol accumulation, but the xylose consumption rate and fermentation rate were found to be very slow (FIG. 5(b)). The yield of xylitol production by the SJ11 YΔGΔ strain was 0.04 (g/g) which is the lowest, indicating that simultaneous deletion of YPR1 and GRE3 was effective in reducing xylitol accumulation (FIG. 5(c) and Table 4). Ethanol production was also enhanced through simultaneous deletion of YPR1 and GRE3. As the fermentation rate increased, the ethanol productivity of the SJ11 YΔGΔ strain showed its maximum production of 18.31 g/L at 288 h. Additionally, the ethanol production yield of the SJ11 YΔGΔ strain was calculated to be 0.46 (g/g), which was an 18% increase compared to the δXI strain and a 7% increase compared to the δXI YΔGΔ strain. Simultaneous deletion of YPR1 and GRE3 in carbon dioxide-fixing yeast resulted in increased fermentation rate, decreased by-product accumulation, and increased ethanol production due to improved CO₂ fixation efficiency, which may serve as a strategy for efficient CO₂ fixation in yeast.

TABLE 4

| | Growth | | | | Xylitol | Glycerol | Ethanol |
|---|---|---|---|---|---|---|---|
| | rate | Product titers (g/L) | | | yield | yield | yield |
| Strains | (g/L-h) | Xylitol | Glycerol | Ethanol | (g/g) | (g/g) | (g/g) |
| δXI | 1.24 ± 0.19 | 3.14 ± 0.07 | 0.99 ± 0.26 | 8.09 ± 1.53 | 0.16 ± 0.03 | 0.05 ± 0.01 | 0.39 ± 0.01 |
| δXI YΔGΔ | 1.04 ± 0.14 | 0.83 ± 0.04 | 0.45 ± 0.10 | 3.55 ± 0.53 | 0.10 ± 0.00 | 0.05 ± 0.01 | 0.43 ± 0.05 |
| SJ11 | 1.30 ± 0.13 | 2.97 ± 0.10 | 0.89 ± 0.12 | 7.91 ± 1.03 | 0.15 ± 0.02 | 0.05 ± 0.00 | 0.41 ± 0.02 |
| SJ11 YΔGΔ | 2.26 ± 0.30 | 1.44 ± 0.07 | 1.88 ± 0.16 | 18.31 ± 0.02 | 0.04 ± 0.00 | 0.05 ± 0.00 | 0.46 ± 0.00 |

While a specific part of the present disclosure has been described in detail above, it is clear for those skilled in the art that this specific description is merely preferred examples, and the scope of the present disclosure is not limited thereby. Accordingly, the substantial scope of the present disclosure will be defined by the appended claims and their equivalents.

### <Accession Number>

Name of Depositor: Korean Collection for Type Cultures (KCTC), Korea Research Institute of Bioscience and Biotechnology
Accession Number: KCTC 15918BP
Date of Deposit: 2024 May 31

What is claimed is:

1. A recombinant Saccharomyces cerevisiae SJ11 strain which is deposited under accession number KCTC 15918BP and has improved CO₂ fixation efficiency based on a xylose isomerase pathway.

2. The recombinant Saccharomyces cerevisiae SJ11 strain of claim 1, wherein the strain comprises cbbL1, cbbS1, and cfxP1 derived from Ralstonia eutropha H16, and HSP60 and HSP10 derived from Saccharomyces cerevisiae D452-2 with their signal peptides removed, introduced into its genome.

3. A recombinant Saccharomyces cerevisiae SJ11 YΔGΔ strain with GRE3 and YPR1 deleted from a genome of the recombinant Saccharomyces cerevisiae SJ11 strain according to claim 1.

4. The recombinant Saccharomyces cerevisiae SJ11 strain according to claim 1, wherein the strain exhibits reduced xylitol accumulation and increased ethanol production.

5. The recombinant Saccharomyces cerevisiae SJ11 YΔGΔ strain according to claim 3, wherein the strain exhibits reduced xylitol accumulation and increased ethanol production.

6. A method of improving ethanol production, comprising culturing the recombinant Saccharomyces cerevisiae SJ11 YΔGΔ strain according to claim 3.
